(19) United States
(12) Patent Application Publication
(10) Pub. No.: US 2005/0106214 A1
(43) Pub. Date: May 19, 2005

(54) Excipients in drug delivery vehicles

(76) Inventor: Guohua Chen, Sunnyvale, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/985,122

(22) Filed: Nov. 10, 2004

Related U.S. Application Data
(60) Provisional application No. 60/519,936, filed on Nov. 14, 2003.

(51) Int. Cl. A61K 9/14

(52) U.S. Cl. 424/426

(57) ABSTRACT

Injectable depot gel compositions and kits that provide an excipient for modulating a release rate and stabilizing beneficial agents are provided. Methods of administering and preparing such systems are also provided. The gel compositions comprise biodegradable, bioerodible polymers and water-immiscible solvents in amounts effective to plasticize the polymers and form gels with the polymers. Suitable excipients include pH modifiers, reducing agents, and antioxidants.

### CROSS-REFERENCE TO RELATED APPLICATIONS

This application claims the benefits of U.S. Provisional Application No. 60/519,936, filed on Nov. 14, 2003, which is incorporated herein by reference.

### FIELD OF THE INVENTION

The present invention relates generally to sustained release depot compositions and kits which provide sustained release of a beneficial agent. The present invention also relates to methods of preparing and administering the compositions.

### BACKGROUND OF THE INVENTION

Biodegradable polymers have been used for many years in medical applications. Illustrative devices composed of the biodegradable polymers include sutures, surgical clips, staples, implants, and drug delivery systems. The majority of these biodegradable polymers have been based upon glycolide, lactide, caprolactone, and copolymers thereof.

Biodegradable polymer formulations for injectable implants have used solvent/plasticizers that are very or relatively soluble in aqueous body fluids to promote rapid solidification of the polymer at the implant site and promote diffusion of drug from the implant. Rapid migration of water into such polymeric implants utilizing water soluble solvents when the implants are placed in the body and exposed to aqueous body fluids presents a serious problem. The rapid water uptake often results in implants having pore structures that are non-homogeneous in size and shape. Typically, the surface pores take on a finger-like pore structure extending for as much as one-third of a millimeter or more from the implant surface into the implant, and such finger-like pores are open at the surface of the implant to the environment of use. The internal pores tend to be smaller and less accessible to the fluids present in the environment of use. The rapid water uptake characteristic often results in uncontrolled release of beneficial agent that is manifested by an initial, rapid release of beneficial agent from the polymer formulation, corresponding to a “burst” of beneficial agent being released from the implant. The burst often results in a substantial portion of the beneficial agent, if not all, being released in a very short time, e.g., hours or 1-2 days. Such an effect can be unacceptable, particularly in those circumstances where a controlled delivery is desired, i.e., delivery of beneficial agent in a controlled manner over a period of greater than two weeks or up to a month, or even up to one year, or where there is a narrow therapeutic window and release of excess beneficial agent can result in adverse consequences to the subject being treated, or where it is necessary to mimic the naturally-occurring daily profile of beneficial agents, such as hormones and the like, in the body of the subject being treated.

Accordingly, when such devices are implanted, the finger-like pores allow very rapid uptake of aqueous body fluids into the interior of the implant with consequent immediate and rapid dissolution of significant quantities of beneficial agent and unimpeded diffusion of beneficial agent into the environment of use, producing the burst effect discussed above.

Furthermore, rapid water uptake can result in premature polymer precipitation such that a hardened implant or one with a hardened skin is produced. The inner pores and much of the interior of the polymer containing beneficial agent are shut off from contact with the body fluids and a significant reduction in the release of beneficial agent can result over a not insignificant period of time (“lag time”). That lag time is undesirable from the standpoint of presenting a controlled, sustained release of beneficial agent to the subject being treated. What one observes, then, is a burst of beneficial agent being released in a short time period immediately after implantation, a lag time in which no or very little beneficial agent is being released, and subsequently continued delivery of beneficial agent (assuming beneficial agent remains after the burst) until the supply of beneficial agent is exhausted.

Various approaches to control burst and modulate and stabilize the delivery of the beneficial agent have been described. The following patents U.S. Pat. Nos. 6,468,961; 6,331,311; 6,130,200; 5,990,194; 5,780,044; 5,733,950; 5,656,297; 5,654,010; 4,985,404 and 4,853,218 and PCT publication WO 98/27962 are believed to be representative and are incorporated herein by reference. Notwithstanding some success, those methods have not been entirely satisfactory for the large number of beneficial agents that would be effectively delivered by implants.

Initial burst release and release rate profile of a drug can be affected by many factors, such as the ratio of polymer to solvent, the molecular weight of the polymer, the water miscibility of the solvent, and properties of the drug particles. Achieving a desired release rate, however, can be inhibited by, in some cases, deterioration of the beneficial agent. Furthermore, when polymeric matrices trap beneficial agents, release of the beneficial agents from inside of the polymer matrices could be predominantly diffusion-controlled before polymer matrices start to degrade significantly, leading to a release rate profile which might not be desirable.

A problem presented by the use of some biodegradable polymers in drug delivery systems is degradation of the polymer resulting in the build-up of, for example, acid by-products within the delivery system. The resulting environments containing products of polymer degradation can be damaging to beneficial agents, such as proteins, peptides, and small molecular drugs.

Another problem presented by the use of some implantable systems is the presence of free radicals and/or peroxides from body fluids. Normal foreign body reactions to, for example, an implantable drug delivery system, also result in the generation of free radicals and peroxides. As such, free radicals and peroxides can diffuse into implanted drug delivery systems, and then be harmful to beneficial agents.

As a result, beneficial agents are susceptible to deterioration from several sources, thereby reducing the overall effectiveness of the dosage forms because not all of the intended beneficial agent may be available to a subject for therapy.

There remains a great need for drug delivery systems which can stabilize beneficial agents which are exposed to damaging microenvironments due to polymer degradation, and/or the presence of undesired free radicals or peroxides. Furthermore, a need continues to exist for modulating release of beneficial agents from drug delivery systems to achieve desirable release rates.

### SUMMARY OF THE INVENTION

Injectable depot gel compositions and kits that release a beneficial agent over both a short duration and a prolonged duration are provided by the present invention. Methods of administering and preparing such compositions are also provided. Compositions in accordance with the present invention include a gel vehicle, a beneficial agent dissolved or dispersed in the gel vehicle, and an excipient. The gel vehicle comprises a bioerodible, biocompatible polymer and a water-immiscible solvent in an amount effective to plasticize the polymer and form a gel with the polymer. In some instances, a component solvent is used along with the water-immiscible solvent. Compositions of the present invention use excipients to modulate release profiles and stabilize beneficial agents. For example, some excipients can offset the effects of degradation of the polymer. Other excipients can offset the effects of peroxides and/or free radicals from body fluids.

An embodiment in accordance with the present invention includes injectable depot gel compositions for the sustained delivery of a beneficial agent comprising: a gel vehicle comprising a bioerodible, biocompatible polymer and a water-immiscible solvent in an amount effective to plasticize the polymer and form a gel therewith; a beneficial agent dissolved or dispersed in the gel vehicle; and an excipient for modulating a release rate and stabilizing the beneficial agent; wherein the sustained delivery occurs during a period of between about twenty-four hours to about twelve months after administration.

Although there are many suitable excipients, examples include pH modifiers, reducing agents, and antioxidants. Embodiments of the present invention may use a single excipient or a combination of excipients.

Excipients that are pH modifiers, include, but are not limited to inorganic salts, such as zinc carbonate, magnesium carbonate, calcium carbonate, magnesium hydroxide, calcium hydrogen phosphate, calcium acetate, calcium hydroxide, calcium lactate, calcium maleate, calcium oleate, calcium oxalate, calcium phosphate, magnesium acetate, magnesium hydrogen phosphate, magnesium phosphate, magnesium lactate, magnesium maleate, magnesium oleate, magnesium oxalate, zinc acetate, zinc hydrogen phosphate, zinc phosphate, zinc lactate, zinc maleate, zinc oleate, zinc oxalate, and combinations thereof. Excipients that are reducing agents can be cysteine or methionine. Antioxidants used as excipients can be selected from the group consisting of: d-alpha tocopherol acetate, dl-alpha tocopherol, ascorbyl palmitate, butylated hydroxyanidole, ascorbic acid, butylated hydroxyanisole, butylatedhydroxyquinone, butylhydroxyanisol, hydroxycomarin, butylated hydroxytoluene, cephalm, ethyl gallate, propyl gallate, octyl gallate, lauryl gallate, propylhydroxybenzoate, trihydroxybutylrophenone, dimethylphenol, diterlbulylphenol, vitamin E, lecithin, ethanolamine, and combinations thereof.

With reference to the excipient, compositions of the present invention can comprise between about 0.01% and about 50% by weight; between about 0.05% and about 40% by weight; or between about 0.1% and about 30% by weight. In addition, the ratio between the excipient and the beneficial agent can be between about 0.1:99.9 and about 99:1, preferably the ratio is between about 1:99 and about 60:40.

Water-immiscible solvents of the invention can have miscibilities in water of less than or equal to about 7 weight % at 25° C. Furthermore, compositions can be free of solvents having a miscibility in water that is greater than 7 weight % at 25° C. Solvents can be selected from the group consisting of: an aromatic alcohol, lower alkyl esters of aryl acids, lower aralkyl esters of aryl acids; aryl ketones, aralkyl ketones, lower alkyl ketones, lower alkyl esters of citric acid, and combinations thereof. Other solvents useful in the present invention are benzyl alcohol, benzyl benzoate, ethyl benzoate, and triacetin.

Some embodiments of the present invention comprise a component solvent selected from the group consisting of: triacetin, diacetin, tributyrin, triethyl citrate, tributyl citrate, acetyl triethyl citrate, acetyl tributyl citrate, triethylglycerides, triethyl phosphate, diethyl phthalate, diethyl tartrate, mineral oil, polybutene, silicone fluid, glylcerin, ethylene glycol, polyethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, methyl acetate, ethyl acetate, methyl ethyl ketone, dimethylformamide, dimethyl sulfoxide, tetrahydrofuran, caprolactam, decylmethylsulfoxide, oleic acid, and 1-dodecylazacyclo-heptan-2-one, and combinations thereof.

Polymers used in accordance with the invention can be selected from the group consisting of: polylactides, polyglycolides, poly(caprolactone), polyanhydrides, polyamines, polyesteramides, polyorthoesters, polydioxanones, polyacetals, polyketals, polycarbonates, polyphosphoesters, polyesters, polybutylene terephthalate, polyorthocarbonates, polyphosphazenes, succinates, poly(malic acid), poly(amino acids), polyvinylpyrrolidone, polyethylene glycol, polyhydroxycellulose, polysaccharides, chitin, chitosan, hyaluronic acid, and copolymers, terpolymers and mixtures thereof. Lactic acid-based polymers, preferably copolymers of lactic acid and glycolic acid (PLGA), including poly(D,L-lactide-co-glycolide) and poly(L-lactide-co-glycolide) can be used in the present invention. In some embodiments, the PLGA polymers have a weight average molecular weights of between about 3,000 to about 120,000 and monomer ratios of lactic acid to glycolic acid of between about 50:50 to about 100:0. Caprolactone-based polymers can also be used in the present invention.

Other embodiments of the present invention comprise between about 5 weight % and about 90 weight % of the polymer, between about 25 weight % and about 80 weight %, or between about 35 weight % and about 75 weight %. In terms of the ratio between the polymer and the solvent, some ratios may be between about 5:95 and about 90:10, others may be between about 20:80 and about 80:20, still others may be between about 30:70 and about 75:25.

In accordance with the present invention, compositions can further comprise at least one of the following: an emulsifying agent, a pore former, a solubility modulator for the anesthetic, and an osmotic agent.

With respect to beneficial agents, compositions can comprise from about 0.1% to about 50% beneficial agent by weight, from about 0.5% to about 40%, or from about 1% to about 30%. Average particle sizes of the beneficial agents can be less than about 250 μm, between about 5 μm and 250 μm, between about 20 μm and about 125 μm, or between about 38 μm and about 63 μm.

Beneficial agents can be selected from the group consisting of: a protein, a peptide, a drug, and combinations thereof. For example, when the beneficial agent comprises a protein, the protein can be selected from the group consisting of: human growth hormone, interferon alpha-2a, interferon alpha-2b, EPO, methionine-human growth hormone, des-phenylalanine human growth hormone, consensus interferon, and combinations thereof. When the beneficial agent comprises a drug, the drug can be bupivacaine or praclitaxil. Beneficial agents that are peptides can include leuprolide or desmopressin.

In one embodiment of the present invention methods of preparing an injectable depot gel composition for sustained delivery of a beneficial agent to a subject over a duration of between about twenty-four hours to about twelve months is provided, the methods comprising: mixing a bioerodible, biocompatible polymer and an effective plasticizing amount of a water-immiscible solvent to form a gel vehicle; mixing a beneficial agent into the gel vehicle; mixing an excipient for modulating a release rate into the gel vehicle; and stabilizing the beneficial agent wherein the presence of the excipient offsets the effects of degradation of the polymer. Methods can further comprise premixing the excipient with the beneficial agent before mixing the excipient and the beneficial agent into the gel vehicle. On the other hand, methods can further comprise loading the excipient and the beneficial agent separately into the gel vehicle. The excipient can be dissolved or dispersed in the gel vehicle.

Other methods of the present invention include preparing an injectable depot gel composition for sustained delivery of a beneficial agent to a subject over a duration of between about twenty-four hours to about twelve months is provided, the methods comprising: mixing a bioerodible, biocompatible polymer and an effective plasticizing amount of a water-immiscible solvent to form a gel vehicle; mixing a beneficial agent into the gel vehicle; mixing an excipient for modulating a release rate into the gel vehicle; and stabilizing the beneficial agent wherein the presence of the excipient offsets peroxides or free radicals or both found in body fluid.

Another embodiment of the invention includes methods of administering an injectable depot composition for sustained release of a beneficial agent over a duration of between about twenty-four hours to about twelve months comprising: administering a composition comprising a gel vehicle comprising a bioerodible, biocompatible polymer and an effective plasticizing amount of a water-immiscible solvent to form a gel vehicle; a beneficial agent dissolved or dispersed in the gel vehicle; and an excipient for modulating a release rate and stabilizing the beneficial agent. The compositions can be administered once. On the other hand, compositions can be administered repeatedly. The compositions can be delivered locally or systemically. In addition, the compositions can be delivered to multiple sites on the subject.

Still another embodiment of the invention includes kits for administration of a sustained delivery of a beneficial agent for a period of between about twenty-four hours to about twelve months after administration, the kits comprising: a gel vehicle comprising a bioerodible, biocompatible polymer and a water-immiscible solvent, in an amount effective to plasticize the polymer and form a gel therewith; a beneficial agent dissolved or dispersed in the gel vehicle; an excipient for modulating a release rate, wherein the excipient stabilizes the beneficial agent by offsetting the effects of degradation of the polymer; and optionally, one or more of the following: an emulsifying agent; a pore former; a solubility modulator for the anesthetic, optionally associated with the beneficial agent; and an osmotic agent; wherein at the least anesthetic agent, optionally associated with the solubility modulator, is maintained separated from the solvent until the time of administration of the anesthetic agent to the subject.

Yet another embodiment of the invention includes kits for administration of a sustained delivery of a beneficial agent for a period of between about twenty-four hours to about twelve months after administration, the kits comprising: a gel vehicle comprising a bioerodible, biocompatible polymer and a water-immiscible solvent, in an amount effective to plasticize the polymer and form a gel therewith; a beneficial agent dissolved or dispersed in the gel vehicle; an excipient for modulating a release rate, wherein the excipient stabilizes the beneficial agent by offsetting the effects of degradation of the polymer; and optionally, one or more of the following: an emulsifying agent; a pore former; a solubility modulator for the anesthetic, optionally associated with the beneficial agent; and an osmotic agent; wherein at the least anesthetic agent, optionally associated with the solubility modulator, is maintained separated from the solvent until the time of administration of the anesthetic agent to the subject.

These and other embodiments will readily occur to those or ordinary skill in the art in view of the disclosure herein.

### BRIEF DESCRIPTION OF THE DRAWINGS

The foregoing and other objects, features and advantages of the present invention will be more readily understood upon reading the following detailed description in conjunction with the drawings as described hereinafter.

FIG. 1 is a graph illustrating the in vivo release profile of bupivacaine base obtained from depot formulations of the present invention (formulations 1-2).

FIG. 2 is a graph illustrating the in vivo release profile of bupivacaine hydrochloride obtained from depot formulations of the present invention (formulations 3-5).

FIG. 3 is a graph illustrating the in vivo release profile of hGH obtained from a depot formulation of the present invention (formulations 6-8).

### DETAILED DESCRIPTION

It has been discovered that in certain systems, beneficial agents of injectable depot compositions can be stabilized and their release modulated in the presence of an excipient.

Compositions of the present invention use excipients to offset the effects of polymer degradation and modulate release profiles. Although there are many suitable excipients, examples include pH modifiers and antioxidants, such as reducing agents and free radical scavengers.

Modifiers of pH include, but are not limited to, inorganic and organic salts including zinc carbonate, magnesium carbonate, calcium carbonate, magnesium hydroxide, calcium hydrogen phosphate, calcium acetate, calcium hydroxide, calcium lactate, calcium maleate, calcium oleate, calcium oxalate, calcium phosphate, magnesium acetate, magnesium hydrogen phosphate, magnesium phosphate, magnesium lactate, magnesium maleate, magnesium oleate, magnesium oxalate, zinc acetate, zinc hydrogen phosphate, zinc phosphate, zinc lactate, zinc maleate, zinc oleate, zinc oxalate, and combinations thereof. Reducing agents include, but are not limited to cysteine or methionine. Antioxidants include, but are not limited to, d-alpha tocopherol acetate, dl-alpha tocopherol, ascorbyl palmitate, butylated hydroxyanidole, ascorbic acid, butylated hydroxyanisole, butylatedhydroxyquinone, butylhydroxyanisol, hydroxycomarin, butylated hydroxytoluene, cephalm, ethyl gallate, propyl gallate, octyl gallate, lauryl gallate, propylhydroxybenzoate, trihydroxybutylrophenone, dimethylphenol, diterlbulylphenol, vitamin E, lecithin, and ethanolamine.

Compositions contemplated by the present invention include those that incorporate excipients such as inorganic salts, e.g., magnesium carbonate or zinc carbonate, which can (1) balance the local pH within the depot formulation to protect the beneficial agent from a low pH due to the polymer degradation and (2) modulate the release rate profile through dynamically creating a micro-porous structure in the polymer. Due to the weak base nature of some of the inorganic salts selected, the local acidic pH in the depot microenvironment caused by degradation of the polymer can be balanced. The beneficial agents, especially proteins, peptides, and drugs, therefore, can be protected from the damaging effects of a low pH. In addition, without intending to be bound by theory, it is thought that when particles of excipients such as inorganic salts leave polymeric matrices by dissolution in water, the void space originally occupied by the salt would dynamically create a microporous structure. The pore size and density can be controlled by the starting materials and level of loading. A desirable release profile, thus, may be programmable.

Further, many small molecular drugs are present in different forms depending on the pH of the environment the drugs are exposed to. For example, a small molecular drug may possess a positive charge at low pH, a negative charge at relatively high pH, and no charge at an intermediate pH. By changing the local pH, therefore, the hydrophilic-hydrophobic property of the drug and the solubility of the drug in the matrices might be easily tailored. Thus, the initial burst release and release rate profile of the beneficial agent from the depot can be modulated. It is known that the release rate profile of the active agent from the depot can be highly dependent on the hydrophilic-hydrophobic property of the drug. Since the hydrophilic-hydrophobic property of the drug can be easily tailored by its chemical form and in many cases by the local pH, the approach in this invention might not require any additional formulating materials in the drug particle formulation to modulate solubility of the drug, thus, making the drug formulation much simpler.

Further, many small molecular drugs contain functional moieties such as amine, hydroxyl group which are susceptible to oxidation when peroxide or free radicals are present. When oxidized, the drugs can lose their activity and/or cause some undesired side effects. By incorporating antioxidants, such as, but not limited to, reducing agents or free radical scavengers, the integrity of the drugs can be protected from the attack of the peroxide or free radicals or both that diffuse into the gel vehicle from the body fluid or that result from the normal foreign body reactions to the implants. In addition, without intending to be bound by theory, it is thought that when particles of excipients such as solid reducing agents, antioxidants, and free radical scavengers, or dispersed droplets of excipients such as solid reducing agents, antioxidants, and free radical scavengers leave polymeric matrices by diffusion, the void space originally occupied by the excipients would dynamically create a microporous structure. The pore size and density can be controlled by the starting materials and level of loading. A desirable release profile, thus, may be programmable.

Biological active agents such as proteins, peptides, monoclonal antibodies etc. are generally susceptible to oxidation when peroxide or free radicals are present. When oxidized, the biological active agents could lose their activities and/or cause some undesired side effects such as immune reactions. By incorporating reducing agents, antioxidants, or free radical scavengers, the integrity of the agents can be protected from the attack of the peroxide and/or free radicals that diffuse in from the body fluid or that result from the normal foreign body reactions to the implants. In addition, without intending to be bound by theory, it is thought that when particles of excipients such as solid reducing agents, antioxidants, and free radical scavengers, or dispersed droplets of excipients such as solid reducing agents, antioxidants, free radical scavengers, leave polymeric matrices by diffusion, the void space originally occupied by the excipients would dynamically create a microporous structure. The pore size and density can be controlled by the starting materials and level of loading. A desirable release profile, thus, may be programmable.

Compositions according to the present invention incorporate excipients such as antioxidants, reducing agents, and/or free radical scavengers which target, for example, free radicals and peroxides that are diffused into the gel vehicle from the body fluid or that result from the normal foreign body reaction to the implants.

Incorporation of the excipients into the gel vehicles can be done, for example, by directly incorporating, or pre-mixing, the excipient into the drug particles during the drug particle formulation processing. On the other hand, the excipient and drug can be loaded separately into the gel vehicle. Excipients, like beneficial agents, can be dissolved or dispersed in the gel vehicle.

Definitions

In describing and claiming the present invention, the following terminology will be used in accordance with the definitions set out below.

The singular forms “a,” “an” and “the” include plural referents unless the context clearly dictates otherwise. Thus, for example, reference to “a solvent” includes a single solvent as well as a mixture of two or more different solvents, reference to “an anesthetic” includes a single anesthetic as well as two or more different anesthetics in combination, and the like.

Reference to the “effects of degradation of the polymer” refers to, without limitation, those by-products which result from breakdown of the biodegradable polymer. Such by-products can include acid by-products, such as lactic acid and glycolic acid, for example, when PLGA is used. In addition, by-products such as oxides, peroxides, and free radicals may be present. By reference to “offsetting the effects of degradation,” therefore, it is meant that by-products are prevented from damaging the beneficial agents. For example, excipients comprising salts can neutralize acid by-products. Excipients comprising reducing agents inhibit peroxides, and likewise, antioxidants prevent oxides from degrading the beneficial agents.

Reference to the “peroxides or free radicals or both” refers to, without limitation, those peroxides and/or free radicals that are present in body fluid that can be harmful to beneficial agents. For example, normal foreign body reaction to, for example, implants, generates free radicals and peroxides that can make their way into an implant and degrade beneficial agents. Other peroxides and free radicals are the result of normal functions of the body and yet still present a harm to beneficial agents.

The term “excipient” means any useful ingredient in the formulation aside from the beneficial agent or the materials used to form the gel vehicle. Excipients useful for modulating a release rate and stabilizing the beneficial agent include pH modifiers, reducing agents, antioxidants, and free radical scavengers.

The term “AUC” means the area under the curve obtained from an in vivo assay in a subject by plotting blood plasma concentration of the beneficial agent in the subject against time, as measured from the time of implantation of the composition, to a time “t” after implantation. The time t will correspond to the delivery period of beneficial agent to a subject.

The term “burst index” means, with respect to a particular composition intended for systemic delivery of a beneficial agent, the quotient formed by dividing (i) the AUC calculated for the first time period after implantation of the composition into a subject divided by the number of hours in the first time period (t₁), by (ii) the AUC calculated for the time period of delivery of beneficial agent, divided by the number of hours in the total duration of the delivery period (t₂). For example the burst index at 24 hours is the quotient formed by dividing (i) the AUC calculated for the first twenty-four hours after implantation of the composition into a subject divided by the number 24, by (ii) the AUC calculated for the time period of delivery of beneficial agent, divided by the number of hours in the total duration of the delivery period.

The phrase “dissolved or dispersed” is intended to encompass all means of establishing a presence of beneficial agent and/or an excipient in the gel composition and includes dissolution, dispersion, suspension and the like.

The term “systemic” means, with respect to delivery or administration of a beneficial agent to a subject, that the beneficial agent is detectable at a biologically-significant level in the blood plasma of the subject.

The term “local” means, with respect to delivery or administration of a beneficial agent to a subject, that the beneficial agent is delivered to a localized site in the subject but is not detectable at a biologically significant level in the blood plasma of the subject.

The term “gel vehicle” means the composition formed by mixture of the polymer and solvent in the absence of the beneficial agent.

The terms “short period” or “short duration” are used interchangeably and refer to a period of time over which release of a beneficial agent from the depot gel composition of the invention occurs, which will generally be equal to or less than two weeks, preferably about 24 hours to about 2 weeks, preferably about 10 days or shorter; preferably about 7 days or shorter, more preferably about 3 days to about 7 days.

The term “prolonged period” or “prolonged duration” means a period of time over which release of a beneficial agent from the implant of the invention occurs, which will generally be about one week or longer, preferably about 30 days or longer, and more preferably one year.

The term “initial burst” means, with respect to a particular composition of this invention, the quotient obtained by dividing (i) the amount by weight of beneficial agent released from the composition in a predetermined initial period of time after implantation, by (ii) the total amount of beneficial agent that is to be delivered from an implanted composition. It is understood that the initial burst may vary depending on the shape and surface area of the implant. Accordingly, the percentages and burst indices associated with initial burst described herein are intended to apply to compositions tested in a form resulting from dispensing of the composition from a standard syringe.

The term “solubility modulator” means, with respect to the beneficial agent, an agent that will alter the solubility of the beneficial agent, with reference to polymer solvent or water, from the solubility of beneficial agent in the absence of the modulator. The modulator may enhance or retard the solubility of the beneficial agent in the solvent or water. However, in the case of beneficial agents that are highly water soluble, the solubility modulator will generally be an agent that will retard the solubility of the beneficial agent in water. The effects of solubility modulators of the beneficial agent may result from interaction of the solubility modulator with the solvent, or with the beneficial agent itself, such as by the formation of complexes, or with both. For the purposes hereof, when the solubility modulator is “associated” with the beneficial agent, all such interactions or formations as may occur are intended. Solubility modulators may be mixed with the beneficial agent prior to its combination with the viscous gel or may be added to the viscous gel prior to the addition of the beneficial agent, as appropriate.

The terms “subject” and “patient” mean, with respect to the administration of a composition of the invention, an animal or a human being.

Since all solvents, at least on a molecular level, will be soluble in water (i.e., miscible with water) to some very limited extent, the term “immiscible” as used herein means that 7% or less by weight, preferably 5% or less, of the solvent is soluble in or miscible with water. For the purposes of this disclosure, solubility values of solvent in water are considered to be determined at 25° C. Since it is generally recognized that solubility values as reported may not always be conducted at the same conditions, solubility limits recited herein as percent by weight miscible or soluble with water as part of a range or upper limit may not be absolute. For example, if the upper limit on solvent solubility in water is recited herein as “7% by weight,” and no further limitations on the solvent are provided, the solvent “triacetin,” which has a reported solubility in water of 7.17 grams in 100 ml of water, is considered to be included within the limit of 7%. A solubility limit in water of less than 7% by weight as used herein does not include the solvent triacetin or solvents having solubilities in water equal to or greater than triacetin.

The term “bioerodible” refers to a material that gradually decomposes, dissolves, hydrolyzes and/or erodes in situ. Generally, the “bioerodible” polymers herein are polymers that are hydrolyzable, and bioerode in situ primarily through hydrolysis.

The polymer, solvent and other agents of the invention must be “biocompatible”; that is they must not cause irritation, inflammation or necrosis in the environment of use. The environment of use is a fluid environment and may comprise a subcutaneous, intramuscular, intravascular (high/low flow), intramyocardial, adventitial, intratumoral, or intracerebral portion, wound sites, tight joint spaces or body cavity of a human or animal.

The term “alkyl” as used herein refers to a saturated hydrocarbon group typically although not necessarily containing 1 to about 30 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 12 carbon atoms. The term “lower alkyl” intends an alkyl group of 1 to 6 carbon atoms, preferably 1 to 4 carbon atoms. “Substituted alkyl” refers to alkyl substituted with one or more substituent groups, and the terms “heteroatom-containing alkyl” and “heteroalkyl” refer to alkyl in which at least one carbon atom is replaced with a heteroatom. If not otherwise indicated, the terms “alkyl” and “lower alkyl” include linear, branched, cyclic, unsubstituted, substituted, and/or heteroatom-containing alkyl or lower alkyl.

The term “aryl” as used herein, and unless otherwise specified, refers to an aromatic substituent containing a single aromatic ring or multiple aromatic rings that are fused together, linked covalently, or linked to a common group such as a methylene or ethylene moiety. Preferred aryl groups contain one aromatic ring or two fused or linked aromatic rings, e.g., phenyl, naphthyl, biphenyl, diphenylether, diphenylamine, benzophenone, and the like, and most preferred aryl groups are monocyclic. “Substituted aryl” refers to an aryl moiety substituted with one or more substituent groups, and the terms “heteroatom-containing aryl” and “heteroaryl” refer to aryl in which at least one carbon atom is replaced with a heteroatom. Unless otherwise indicated, the term “aryl” includes heteroaryl, substituted aryl, and substituted heteroaryl groups.

The term “aralkyl” refers to an alkyl group substituted with an aryl group, wherein alkyl and aryl are as defined above. The term “heteroaralkyl” refers to an alkyl group substituted with a heteroaryl group. Unless otherwise indicated, the term “aralkyl” includes heteroaralkyl and substituted aralkyl groups as well as unsubstituted aralkyl groups. Generally, the term “aralkyl” herein refers to an aryl-substituted lower alkyl group, preferably a phenyl substituted lower alkyl group such as benzyl, phenethyl, 1-phenylpropyl, 2-phenylpropyl, and the like.

I. Injectable Depot Compositions:

In contrast to prior polymer-based injectable depots, depots of the present invention use an excipient which modulates a release rate as well as stabilizes the beneficial agent by offsetting effects of degradation of the polymer. Injectable depot compositions for delivery of beneficial agents over a prolonged period of time may be formed as viscous gels prior to injection of the depot into a subject. The viscous gel supports dispersed beneficial agent to provide appropriate delivery profiles, which include those having low initial burst, of the beneficial agent as the beneficial agent is released from the depot over time.

Typically, the viscous gel will be injected from a standard hypodermic syringe that has been pre-filled with the beneficial agent-viscous gel composition to form the depot. It is often preferred that injections take place using the smallest size needle (i.e., smallest diameter) to reduce discomfort to the subject when the injection takes place through the skin and into subcutaneous tissue. It is desirable to be able to inject gels through needles ranging from 16 gauge and higher, preferably 20 gauge and higher, more preferably 22 gauge and higher, even more preferably 24 gauge and higher. With highly viscous gels, i.e., gels having a viscosity of about 200 poise or greater, injection forces to dispense the gel from a syringe having a needle in the 20-30 gauge range may be so high as to make the injection difficult or reasonably impossible when done manually. At the same time, the high viscosity of the gel is desirable to maintain the integrity of the depot after injection and during the dispensing period and also facilitate desired suspension characteristics of the beneficial agent in the gel.

The depot gel composition described herein exhibits reduced viscosity when subjected to shear force. The extent of the reduction is in part a function of the shear rate of the gel when subjected to the shearing force, the molecular weight of the polymer and the polydispersity of the polymer matrix. When the shearing force is removed, the viscosity of the depot gel composition returns to a viscosity at or near that which it displayed prior to being subjected to the shearing force. Accordingly, the depot gel composition may be subjected to a shearing force when injected from a syringe which temporarily reduces its viscosity during the injection process. When the injection process is completed, the shearing force is removed and the gel returns very near to its previous state.

Excipients

As discussed above, excipients useful for modulating a release rate and stabilizing the beneficial agent include any useful ingredient in the formulation aside from the beneficial agent or the materials used to form the gel vehicle. Excipients useful for modulating a release rate and stabilizing the beneficial agent include, for example, pH modifiers, reducing agents, antioxidants, and free radical scavengers.

Modifiers of pH include, but are not limited to, inorganic and organic salts including zinc carbonate, magnesium carbonate, calcium carbonate, magnesium hydroxide, calcium hydrogen phosphate, calcium acetate, calcium hydroxide, calcium lactate, calcium maleate, calcium oleate, calcium oxalate, calcium phosphate, magnesium acetate, magnesium hydrogen phosphate, magnesium phosphate, magnesium lactate, magnesium maleate, magnesium oleate, magnesium oxalate, zinc acetate, zinc hydrogen phosphate, zinc phosphate, zinc lactate, zinc maleate, zinc oleate, zinc oxalate, and combinations thereof. Reducing agents include, but are not limited to cysteine or methionine. Antioxidants include, but are not limited to, d-alpha tocopherol acetate, dl-alpha tocopherol, ascorbyl palmitate, butylated hydroxyanidole, ascorbic acid, butylated hydroxyanisole, butylatedhydroxyquinone, butylhydroxyanisol, hydroxycomarin, butylated hydroxytoluene, cephalm, ethyl gallate, propyl gallate, octyl gallate, lauryl gallate, propylhydroxybenzoate, trihydroxybutylrophenone, dimethylphenol, diterlbulylphenol, vitamin E, lecithin, and ethanolamine.

Bioerodible, Biocompatible Polymers

Polymers that are useful in conjunction with the methods and compositions of the invention are bioerodible, i.e., they gradually hydrolyze, dissolve, physically erode, or otherwise disintegrate within the aqueous fluids of a patient's body. Generally, the polymers bioerode as a result of hydrolysis or physical erosion, although the primary bioerosion process is typically hydrolysis.

Such polymers include, but are not limited to, polylactides, polyglycolides, polycaprolactones, polyanhydrides, polyamines, polyurethanes, polyesteramides, polyorthoesters, polydioxanones, polyacetals, polyketals, polycarbonates, polyphosphoesters, polyoxaesters, polyorthocarbonates, polyphosphazenes, succinates, poly(malic acid), poly(amino acids), polyvinylpyrrolidone, polyethylene glycol, polyhydroxycellulose, chitin, chitosan, hyaluronic acid and copolymers, terpolymers and mixtures thereof.

Presently preferred polymers are polylactides, that is, a lactic acid-based polymer that can be based solely on lactic acid or can be a copolymer based on lactic acid and glycolic acid, and which may include small amounts of other comonomers that do not substantially affect the advantageous results that can be achieved in accordance with the present invention. As used herein, the term “lactic acid” includes the isomers L-lactic acid, D-lactic acid, DL-lactic acid and lactide, while the term “glycolic acid” includes glycolide. Most preferred are poly(lactide-co-glycolide)copolymers, commonly referred to as “PLGA.” The polymer may have a monomer ratio of lactic acid/glycolic acid of from about 100:0 to about 15:85, preferably from about 75:25 to about 30:70, more preferably from about 60:40 to about 40:60, and an especially useful copolymer has a monomer ratio of lactic acid/glycolic acid of about 50:50.

As indicated in U.S. Pat. No. 5,242,910, the polymer can be prepared in accordance with the teachings of U.S. Pat. No. 4,443,340. Alternatively, the lactic acid-based polymer can be prepared directly from lactic acid or a mixture of lactic acid and glycolic acid (with or without a further comonomer) in accordance with the techniques set forth in U.S. Pat. No. 5,310,865. The contents of all of these patents are incorporated by reference. Suitable lactic acid-based polymers are available commercially. For instance, 50:50 lactic acid: glycolic acid copolymers having molecular weights of 8,000, 10,000, 30,000 and 100,000 are available from Boehringer Ingelheim (Petersburg, Va.), Medisorb Technologies International L.P. (Cincinatti, Ohio) and Birmingham Polymers, Inc. (Birmingham, Ala.) as described below.

Suitable polymers include, but are not limited to, Poly (D,L-lactide-co-glycolide) (PLGA), available as 50:50 DL-PLG with an inherent viscosity of 0.15 (PLGA-BPI, Birmingham Polymers, Inc., Birmingham, Ala.) and 50:50 Resomer® RG502 (PLGA RG 502), Poly (D,L-lactide) Resomer® L104, PLA-L104, code no. 33007, Poly (D,L-lactide-co-glycolide) 50:50 Resomer® RG502, code 0000366, Poly (D,L-lactide-co-glycolide) 50:50 Resomer® RG502H, PLGA-502H, code no. 260187, Poly (D,L-lactide-co-glycolide) 50:50 Resomer® RG503, PLGA-503, code no. 0080765, Poly (D,L-lactide-co-glycolide) 50:50 Resomer® RG755, PLGA-755, code no. 95037, Poly L-Lactide MW 2,000 (Resomer® L 206, Resomer® L 207, Resomer® L 209, Resomer® L 214); Poly D,L Lactide (Resomer® R 104, Resomer® R 202, Resomer® R 203, Resomer® R 206, Resomer® R 207, Resomer® R 208); Poly L-Lactide-co-D,L-lactide 90:10 (Resomer® LR 209); Poly D-L-lactide-co-glycolide 75:25 (Resomer® RG 752, Resomer® RG 756); Poly D,L-lactide-co-glycolide 85:15 (Resomer® RG 858); Poly L-lactide-co-trimethylene carbonate 70:30 (Resomer® LT 706); Poly dioxanone (Resomer® X 210) (Boehringer Ingelheim Chemicals, Inc., Petersburg, Va.); DL-lactide/glycolide 100:0 (MEDISORB® Polymer 100 DL High, MEDISORB® Polymer 100 DL Low); DL-lactide/glycolide 85/15 (MEDISORB® Polymer 8515 DL High, MEDISORB® Polymer 8515 DL Low); DL-lactide/glycolide 75/25 (MEDISORB® Polymer 7525 DL High, MEDISORB® Polymer 7525 DL Low); DL-lactide/glycolide 65/35 (MEDISORB® Polymer 6535 DL High, MEDISORB® Polymer 6535 DL Low); DL-lactide/glycolide 54/46 (MEDISORB® Polymer 5050 DL High, MEDISORB® Polymer 5050 DL Low); and DL-lactide/glycolide 54/46 (MEDISORB® Polymer 5050 DL 2A(3), MEDISORB® Polymer 5050 DL 3A(3), MEDISORB® Polymer 5050 DL 4A(3)) (Medisorb Technologies International L.P., Cincinnati, Ohio); and Poly D,L-lactide-co-glycolide 50:50; Poly D,L-lactide-co-glycolide 65:35; Poly D,L-lactide-co-glycolide 75:25; Poly D,L-lactide-co-glycolide 85:15; Poly DL-lactide; Poly L-lactide; Poly glycolide; Poly co-caprolactone; Poly DL-lactide-co-caprolactone 25:75; and Poly DL-lactide-co-caprolactone 75:25 (Birmingham Polymers, Inc., Birmingham, Ala.).

The biocompatible bioerodible polymers are present in the gel composition in an amount ranging from about 5 to about 90% by weight, preferably from about 25 to about 80% by weight and typically from about 35 to about 75% by weight of the viscous gel, the viscous gel comprising the combined amounts of the biocompatible polymer and a solvent having a miscibility in water that is less than 7 wt. % at 25° C.

The solvent will be added to polymer in amounts described below, to provide implantable or injectable viscous gels.

Solvents:

The injectable depot compositions of the invention can contain a water-immiscible solvent having a miscibility in water that is less than 7 wt. % at 25° C., in addition to the bioerodible polymer, the excipient, and the beneficial agent. The solvent must be biocompatible, should form a gel, preferably a viscous gel with the polymer, and restrict water uptake into the implant. Suitable solvents will substantially restrict the uptake of water by the implant and, as noted above, may be characterized as immiscible in water, i.e., having a solubility or miscibility in water of at most 7% by weight. Preferably, the water solubility of the aromatic alcohol is 5 wt. % or less, more preferably 3 wt. % or less, and even more preferably 1 wt. % or less. Most preferably, the solubility of the aromatic alcohol in water is equal to or less than 0.5 weight percent. In preferred embodiments, the solvent is selected from the group consisting of an aromatic alcohol, esters of aromatic acids, aromatic ketones, and mixtures thereof.

Water miscibility may be determined experimentally as follows: Water (1-5 g) is placed in a tared clear container at a controlled temperature, about 25° C., and weighed, and a candidate solvent is added dropwise. The solution is swirled to observe phase separation. When the saturation point appears to be reached, as determined by observation of phase separation, the solution is allowed to stand overnight and is re-checked the following day. If the solution is still saturated, as determined by observation of phase separation, then the percent (w/w) of solvent added is determined. Otherwise more solvent is added and the process repeated. Solubility or miscibility is determined by dividing the total weight of solvent added by the final weight of the solvent/water mixture. When solvent mixtures are used, they are pre-mixed prior to adding to the water.

The composition may also include, in addition to the water-immiscible solvent(s), one or more additional miscible solvents (“component solvents”), provided that any such additional solvent is other than a lower alkanol. Component solvents compatible and miscible with the primary solvent(s) may have a higher miscibility with water and the resulting mixtures may still exhibit significant restriction of water uptake into the implant. Such mixtures will be referred to as “component solvent mixtures.” Useful component solvent mixtures may exhibit solubilities in water greater than the primary solvents themselves, typically between 0.1 weight percent and up to and including 50 weight percent, preferably up to and including 30 weight percent, and most preferably up to an including 10 weight percent, without detrimentally affecting the restriction of water uptake exhibited by the implants of the invention.

Component solvents useful in component solvent mixtures are those solvents that are miscible with the primary solvent or solvent mixture, and include, but are not limited, to triacetin, diacetin, tributyrin, triethyl citrate, tributyl citrate, acetyl triethyl citrate, acetyl tributyl citrate, triethylglycerides, triethyl phosphate, diethyl phthalate, diethyl tartrate, mineral oil, polybutene, silicone fluid, glycerin, ethylene glycol, polyethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, methyl acetate, ethyl acetate, methyl ethyl ketone, dimethylformamide, dimethyl sulfoxide, tetrahydrofuran, caprolactam, decylmethylsulfoxide, oleic acid, and 1-dodecylazacyclo-heptan-2-one, and mixtures thereof.

The solvent or solvent mixture is capable of dissolving the polymer to form a viscous gel that can maintain particles of the beneficial agent dissolved or dispersed and isolated from the environment of use prior to release. The compositions of the present invention provide implants having a low burst index. Water uptake is controlled by the use of a solvent or component solvent mixture that solublizes or plasticizes the polymer but substantially restricts uptake of water into implant.

The solvent or solvent mixture is typically present in an amount of from about 95 to about 5% by weight, preferably about 75 to about 15% by weight, and most preferably about 65% to about 20% by weight of the viscous gel. In an especially preferred embodiment, the solvent is selected from an aromatic alcohol, lower alkyl and aralkyl esters of benzoic acid. Presently, the most preferred solvents are benzyl benzoate (“BB”), benzyl alcohol (“BA”), ethyl benzoate (“EB”), mixtures of BB and BA, mixtures of BB and ethanol, and mixtures of BB and EB.

Ratios of polymer to solvent include between about 5:95 and about 90:10; preferably between about 20:80 and about 80:20; and more preferably between about 30:70 and about 75:25.

Beneficial Agents:

The beneficial agent can be any physiologically or pharmacologically active substance or substances optionally in combination with pharmaceutically acceptable carriers and additional ingredients such as antioxidants, stabilizing agents, permeation enhancers, etc. that do not substantially adversely affect the advantageous results that can be attained by the present invention. The beneficial agent may be any of the agents which are known to be delivered to the body of a human or an animal and that are preferentially soluble in water rather than in the polymer-dissolving solvent. These agents include drug agents, medicaments, vitamins, nutrients, or the like. Included among the types of agents which meet this description are lower molecular weight compounds, proteins, peptides, genetic material, nutrients, vitamins, food supplements, sex sterilants, fertility inhibitors and fertility promoters.

Drug agents which may be delivered by the present invention include drugs which act on the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system and the central nervous system. Suitable agents may be selected from, for example, proteins, enzymes, hormones, polynucleotides, nucleoproteins, polysaccharides, glycoproteins, lipoproteins, polypeptides, steroids, analgesics, local anesthetics, antibiotic agents, chemotherapeutic agents, immunosuppressive agents, anti-inflammatory agents including anti-inflammatory corticosteroids, antiproliferative agents, antimitotic agents, angiogenic agents, antipsychotic agents, central nervous system (CNS) agents, anticoagulants, fibrinolytic agents, growth factors, antibodies, ocular drugs, and metabolites, analogs (including synthetic and substituted analogs), derivatives (including aggregative conjugates/fusion with other macromolecules and covalent conjugates with unrelated chemical moieties by means known in the art) fragments, and purified, isolated, recombinant and chemically synthesized versions of these species.

Examples of drugs which may be delivered by the composition of the present invention include, but are not limited to bupivacaine, buprenorphine, prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erythrityl tetranitrate, digoxin, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erythromycin, hydrocortisone, hydrocorticosterone acetate, cortisone acetate, dexamethasone and its derivatives such as betamethasone, triamcinolone, methyltestosterone, testosterone, 17-S-estradiol, ethinyl estradiol, ethinyl estradiol 3-methyl ether, prednisolone, 17α-hydroxyprogesterone acetate, 19-nor-progesterone, norgestrel, norethindrone, norethisterone, norethiederone, progesterone, norgesterone, norethynodrel, aspirin, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, chlorpromazine, methyldopa, dihydroxyphenylalanine, theophylline, calcium gluconate, ketoprofen, ibuprofen, cephalexin, erythromycin, haloperidol, zomepirac, ferrous lactate, vincamine, diazepam, phenoxybenzamine, diltiazem, milrinone, mandol, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat, captopril, ramipril, famotidine, nizatidine, sucralfate, etintidine, tetratolol, minoxidil, chlordiazepoxide, diazepam, amitriptyline, imipramine, paliperidone, resperidone, octreotide, alendronate, α-4,β-7 receptor antagonist leukosite and infliximab (Remicade).

Further examples of beneficial agents are proteins and peptides which include, but are not limited to, bone morphogenic proteins, insulin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, bovine somatotropin, porcine somatotropin, oxytocin, vasopressin, GRF, somatostatin, lypressin, pancreozymin, luteinizing hormone, LHRH, LHRH agonists and antagonists, leuprolide, interferons such as interferon alpha-2a, interferon alpha-2b, and consensus interferon, interleukins, growth hormones such as human growth hormone and its derivatives such as methione-human growth hormone and des-phenylalanine human growth hormone, parathyroid hormone, bovine growth hormone and porcine growth hormone, fertility inhibitors such as the prostaglandins, fertility promoters, growth factors such as epidermal growth factors (EGF), platelet-derived growth factors (PDGF), fibroblast growth factors (FGF), transforming growth factors-α (TGF-α), transforming growth factors-β (TGF-β), erythropoietin (EPO), insulin-like growth factor-I (IGF-I), insulin-like growth factor-II (IGF-II), interleukin-1, interleukin-2, interleukin-6, interleukin-8, tumor necrosis factor-α (TNF-α), tumor necrosis factor-β (TNF-β), Interferon-α (INF-α), Interferon-β (IF-β), Interferon-γ (INF-γ), Interferon-ω (INF-ω), colony stimulating factors (CGF), vascular cell growth factor (VEGF), thrombopoietin (TPO), stromal cell-derived factors (SDF), placenta growth factor (P1GF), hepatocyte growth factor (HGF), granulocyte macrophage colony stimulating factor (GM-CSF), glial-derived neurotropin factor (GDNF), granulocyte colony stimulating factor (G-CSF), ciliary neurotropic factor (CNTF), bone growth factor, transforming growth factor, bone morphogeneic proteins (BMP), coagulation factors, human pancreas hormone releasing factor, analogs and derivatives of these compounds, and pharmaceutically acceptable salts of these compounds, or their analogs or derivatives.

The present invention also finds application with chemotherapeutic agents for the local application of such agents to avoid or minimize systemic side effects. Gels of the present invention containing chemotherapeutic agents may be injected directly into the tumor tissue for sustained delivery of the chemotherapeutic agent over time. In some cases, particularly after resection of the tumor, the gel may be implanted directly into the resulting cavity or may be applied to the remaining tissue as a coating. In cases in which the gel is implanted after surgery, it is possible to utilize gels having higher viscosities since they do not have to pass through a small diameter needle. Representative chemotherapeutic agents that may be delivered in accordance with the practice of the present invention include, for example, carboplatin, cisplatin, paclitaxel, BCNU, vincristine, camptothecin, etopside, cytokines, ribozymes, interferons, oligonucleotides and oligonucleotide sequences that inhibit translation or transcription of tumor genes, functional derivatives of the foregoing, and generally known chemotherapeutic agents such as those described in U.S. Pat. No. 5,651,986. The present application has particular utility in the sustained delivery of water soluble chemotherapeutic agents, such as for example cisplatin and carboplatin and the water soluble derivatives of paclitaxel. Those characteristics of the invention that minimize the burst effect are particularly advantageous in the administration of water soluble beneficial agents of all kinds, but particularly those compounds that are clinically useful and effective but may have adverse side effects.

To the extent not mentioned above, the beneficial agents described in aforementioned U.S. Pat. No. 5,242,910 can also be used. One particular advantage of the present invention is that materials, such as proteins, as exemplified by the enzyme lysozyme, and cDNA, and DNA incorporated into vectors both viral and nonviral, which are difficult to microencapsulate or process into microspheres can be incorporated into the compositions of the present invention without the level of degradation caused by exposure to high temperatures and denaturing solvents often present in other processing techniques.

The beneficial agent is preferably incorporated into the viscous gel formed from the polymer and the solvent in the form of particles typically having an average particle size of from less than 250 microns, about 5 to about 250 microns, preferably from about 20 to about 125 microns and often from 38 to 68 microns.

To form a suspension or dispersion of particles of the beneficial agent in the viscous gel formed from the polymer and the solvent, any conventional low shear device can be used such as a Ross double planetary mixer at ambient conditions. In this manner, efficient distribution of the beneficial agent can be achieved substantially without degrading the beneficial agent.

The beneficial agent is typically dissolved or dispersed in the composition in an amount of from about 0.1% to about 50% by weight, preferably in an amount of from about 1% to about 30%, more preferably in an amount of about 2% to about 20%, and often 2 to 10% by weight of the combined amounts of the polymer mixture, solvent, and beneficial agent. Depending on the amount of beneficial agent present in the composition, one can obtain different release profiles and burst indices. More specifically, for a given polymer and solvent, by adjusting the amounts of these components and the amount of the beneficial agent, one can obtain a release profile that depends more on the degradation of the polymer than the diffusion of the beneficial agent from the composition or vice versa. In this respect, at lower beneficial agent loading rates, one generally obtains a release profile reflecting degradation of the polymer wherein the release rate increases with time. At higher loading rates, one generally obtains a release profile caused by diffusion of the beneficial agent wherein the release rate decreases with time. At intermediate loading rates, one obtains combined release profiles so that if desired, a substantially constant release rate can be attained. In order to minimize burst, loading of beneficial agent on the order of 30% or less by weight of the overall gel composition, i.e., polymer, solvent and beneficial agent, is preferred, and loading of 20% or less is more preferred.

Release rates and loading of beneficial agent will be adjusted to provide for therapeutically effective delivery of the beneficial agent over the intended sustained delivery period. Preferably, the beneficial agent will be present in the polymer gel at concentrations that are above the saturation concentration of beneficial agent in water to provide a drug reservoir from which the beneficial agent is dispensed. While the release rate of beneficial agent depends on the particular circumstances, such as the beneficial agent to be administered, release rates on the order of from about 0.1 micrograms/day to about 10 milligrams/day, preferably from about 1 microgram/day to about 5 milligrams per day, more preferably from about 10 micrograms/day to about 1 milligram/day, for periods of from about 24 hours to about 360 days, preferably 24 hours to about 180 days, more preferably 24 hours to about 120 days, often 3 days to about 90 days can be obtained. Further, the dose of beneficial agent may be adjusted by adjusting the amount of depot gel injected. Greater amounts may be delivered if delivery is to occur over shorter periods. Generally, higher release rate is possible if a greater burst can be tolerated. In instances where the gel composition is surgically implanted, or used as a “leave behind” depot when surgery to treat the disease state or another condition is concurrently conducted, it is possible to provide higher doses that would normally be administered if the implant was injected. Further, the dose of beneficial agent may be controlled by adjusting the volume of the gel implanted or the injectable gel injected. Preferably, the system releases 40% or less by weight of the beneficial agent present in the viscous gel within the first 24 hours after implantation in the subject. More preferably, 30% or less by weight of the beneficial agent will be released within the first 24 hours after implantation, and the implanted composition has a burst index of 12 or less, preferably 8 or less.

Optional Additional Components:

Other components may be present in the gel composition, to the extent they are desired or provide useful properties to the composition, such as polyethylene glycol, hydroscopic agents, stabilizing agents, pore forming agents, thixotropic agents and others. When the composition includes a peptide or a protein that is soluble in or unstable in an aqueous environment, it may be highly desirable to include a solubility modulator that may, for example, be a stabilizing agent, in the composition. Various modulating agents are described in U.S. Pat. Nos. 5,654,010 and 5,656,297, the disclosures of which are incorporated herein by reference. In the case of hGH, for example, it is preferable to include an amount of a salt of a divalent metal, preferably zinc. Examples of such modulators and stabilizing agents, which may form complexes with the beneficial agent or associate to provide the stabilizing or modulated release effect, include metal cations, preferably divalent, present in the composition as magnesium carbonate, zinc carbonate, calcium carbonate, magnesium acetate, magnesium sulfate, zinc acetate, zinc sulfate, zinc chloride, magnesium chloride, magnesium oxide, magnesium hydroxide, other antacids, and the like. The amounts of such agents used will depend on the nature of the complex formed, if any, or the nature of the association between the beneficial agent and the agent. Molar ratios of solubility modulator or stabilizing agent to beneficial agent of about 100:1 to 1:1, preferably 10:1 to 1:1, typically can be utilized.

Pore forming agents include biocompatible materials that when contacted with body fluids dissolve, disperse or degrade to create pores or channels in the polymer matrix. Typically, organic and non-organic materials that are water soluble such as sugars (e.g., sucrose, dextrose), water soluble salts (e.g., sodium chloride, sodium phosphate, potassium chloride, and sodium carbonate), water soluble solvents such as N-methyl-2-pyrrolidone and polyethylene glycol and water soluble polymers (e.g., carboxymethylcellulose, hydroxypropylcellulose, and the like) can conveniently be used as pore formers. Such materials may be present in amounts varying from about 0.1% to about 100% of the weight of the polymer, but will typically be less than 50% and more typically less than 10-20% of the weight of polymer.

Thixotropic agents include agents that impart thixotropic properties to the polymer gel, such as lower alkanols (e.g. ethanol, isopropanol), and the like. It is to be understood that the thixotropic agent of the present invention does not constitute a mere diluent or a polymer-solvent that reduces viscosity by simply decreasing the concentration of the components of the composition. The use of conventional diluents can reduce viscosity, but can also cause the burst effect mentioned previously when the diluted composition is injected. In contrast, the injectable depot composition of the present invention can be formulated to avoid the burst effect by selecting the thixotropic agent so that once injected into place, the thixotropic agent has little impact on the release properties of the original system. Preferably, the system releases 40% or less by weight of the beneficial agent present in the viscous gel within the first 24 hours after implantation in the subject. More preferably, 30% or less by weight of the beneficial agent will be released within the first 24 hours after implantation, and the implanted composition has a burst index of 12 or less, preferably 8 or less.

II. Utility and Administration:

The means of administration of the implants is not limited to injection, although that mode of delivery may often be preferred. Where the implant will be administered as a leave-behind product, it may be formed to fit into a body cavity existing after completion of surgery or it may be applied as a flowable gel by brushing or palleting the gel onto residual tissue or bone. Such applications may permit loading of beneficial agent in the gel above concentrations typically present with injectable compositions.

To further understand the various aspects of the present invention, the results set forth in the previously described figures were obtained in accordance with the following examples.

### EXAMPLES

Below are several examples of specific embodiments for carrying out the present invention. The examples are offered for illustrative purposes only, and are not intended to limit the scope of the present invention in any way.

### Example 1

Depot Gel Preparation

A gel vehicle for use in an injectable depot of the composition was prepared as follows. A glass vessel was tared on a Mettler PJ3000 top loader balance. Poly (D,L-lactide-co-glycolide) (PLGA), available as 50:50 DL-PLG with an inherent viscosity of 0.15 (PLGA-BPI, Birmingham Polymers, Inc., Birmingham, Ala.) and 50:50 Resomer® RG502 (PLGA RG 502), was weighed into the glass vessel. The glass vessel containing the polymer was tared and the corresponding solvent was added. Amounts expressed as percentages for various polymer/solvent combinations are set forth in Table 1, below. The polymer/solvent mixture was stirred at 250±50 rpm (IKA electric stirrer, IKH-Werke GmbH and Co., Stanfen, Germany) for about 5-10 minutes, resulting in a sticky paste-like substance containing polymer particles. The vessel containing the polymer/solvent mixture was sealed and placed in a temperature controlled incubator equilibrated to 37° C. for 1 to 4 days, with intermittent stirring, depending on solvent and polymer type and solvent and polymer ratios. The polymer/solvent mixture was removed from the incubator when it appeared to be a clear amber homogeneous solution. Thereafter, the mixture was placed in an oven (65° C.) for 30 minutes. It was noted that the PLGA was dissolved in the mixture upon removal from the oven.

Additional depot gel vehicles are prepared with the following solvents or mixtures of solvents: benzyl benzoate (“BB”), benzyl alcohol (“BA”), ethyl benzoate (“EB”), BB/BA, BB/Ethanol, BB/EB and the following polymers: Poly (D,L-lactide) Resomer® L104, PLA-L104, code no. 33007, Poly (D,L-lactide-co-glycolide) 50:50 Resomer® RG502, code 0000366, Poly (D,L-lactide-co-glycolide) 50:50 Resomer® RG502H, PLGA-502H, code no. 260187, Poly (D,L-lactide-co-glycolide) 50:50 Resomer® RG503, PLGA-503, code no. 0080765, Poly (D,L-lactide-co-glycolide) 50:50 Resomer® RG755, PLGA-755, code no. 95037, Poly L-Lactide MW 2,000 (Resomer® L 206, Resomer® L 207, Resomer® L 209, Resomer® L 214); Poly D,L Lactide (Resomer® R 104, Resomer® R 202, Resomer® R 203, Resomer® R 206, Resomer® R 207, Resomer® R 208); Poly L-Lactide-co-D,L-lactide 90:10 (Resomer® LR 209); Poly D-L-lactide-co-glycolide 75:25 (Resomer® RG 752, Resomer® RG 756); Poly D,L-lactide-co-glycolide 85:15 (Resomer® RG 858); Poly L-lactide-co-trimethylene carbonate 70:30 (Resomer® LT 706); Poly dioxanone (Resomer® X 210) (Boehringer Ingelheim Chemicals, Inc., Petersburg, Va.); DL-lactide/glycolide 100:0 (MEDISORB® Polymer 100 DL High, MEDISORB® Polymer 100 DL Low); DL-lactide/glycolide 85/15 (MEDISORB® Polymer 8515 DL High, MEDISORB® Polymer 8515 DL Low); DL-lactide/glycolide 75/25 (MEDISORB® Polymer 7525 DL High, MEDISORB® Polymer 7525 DL Low); DL-lactide/glycolide 65/35 (MEDISORB® Polymer 6535 DL High, MEDISORB® Polymer 6535 DL Low); DL-lactide/glycolide 54/46 (MEDISORB® Polymer 5050 DL High, MEDISORB® Polymer 5050 DL Low); and DL-lactide/glycolide 54/46 (MEDISORB® Polymer 5050 DL 2A(3), MEDISORB® Polymer 5050 DL 3A(3), MEDISORB® Polymer 5050 DL 4A(3)) (Medisorb Technologies International L.P., Cincinnati, Ohio); and Poly D,L-lactide-co-glycolide 50:50; Poly D,L-lactide-co-glycolide 65:35; Poly D,L-lactide-co-glycolide 75:25; Poly D,L-lactide-co-glycolide 85:15; Poly DL-lactide; Poly L-lactide; Poly glycolide; Poly ε-caprolactone; Poly DL-lactide-co-caprolactone 25:75; and Poly DL-lactide-co-caprolactone 75:25 (Birmingham Polymers, Inc., Birmingham, Ala.).

### Example 2

Bupivacaine Base Preparation

Bupivacaine hydrochloride (Sigma-Aldrich Corporation, St. Louis, Mo.) was dissolved in de-ionized (DI) water at a concentration of 40 mg/ml (saturation). A calculated amount of sodium hydroxide (1 N solution) was added to the solution and the pH of the final mixtures was adjusted to 10 to precipitate the BP base. The precipitated product was filtered, and further washed with DI water for at least three times. The precipitated product was dried at approximately 40° C. in vacuum for 24 hours.

### Example 3

Bupivacaine Particle Preparation

Bupivacaine drug particles using bupivacaine hydrochloride (Sigma-Aldrich Corporation, St. Louis, Mo.) or bupivacaine base prepared according example 2 and hydrochloride salt, were prepared as follows. Bupivicaine was grounded and then sieved to a fixed range using 3″ stainless steel sieves. Typical ranges included 25 μm to 38 μm, 38 μm to 63 μm, and 63 μm to 125 μm.

### Example 4

HGH/Zn Complex Preparation

hGH solution (5 mg/ml) solution in water (BresaGen Corporation, Adelaide, Australia) was concentrated to 10 mg/mL using a Concentration/Dialysis Selector diafiltering apparatus. The diafiltered hGH solution was washed with 5 times volume of tris (pH 7.6) and further concentrated to 40 mg/ml solution of hGH in 5 mM TRIS buffer. An equal part of a 27.2 mM zinc (from zinc acetate) in 5 mM TRIS buffer solution was added to yield a final mixture with a 15:1 zinc:hGH mole ratio. This mixture was allowed to complex for approximately one hour at 4° C. This complex was then pre-cooled to −70° C. and lyophilized using a Durastop μP Lyophilizer in accordance with the freezing and drying cycles as described below.

| | |
|---|---|
| Freezing | Ramp down at 2.5 C./min to −30° C. and hold for 30 min |
| cycle | Ramp down at 2.5 C./min to −30° C. and hold for 30 min |
| Drying | Ramp up at 0.5 C./min to 10° C. and hold for 960 min |
| cycle | Ramp up at 0.5 C./min to 20° C. and hold for 480 min |
| | Ramp up at 0.5 C./min to 25° C. and hold for 300 min |
| | Ramp up at 0.5 C./min to 30° C. and hold for 300 min |
| | Ramp up at 0.5 C./min to 5° C. and hold for 5000 min |

### Example 5

Particles of hGH/Zn Complex Preparation

Different particles of hGH/Zn complex were made from those lyophilized hGH/Zn complex prepared in Example 4, either without compression or with compression: 1) the lyophilized hGH/Zn complex was ground without compression using a Waring blender. Particles were collected between a 120-mesh (125 μm) and 400-mesh (38 μm) sieve. 2) The lyophilized hGH/Zn complex was transferred to a 13 mm round compression die and compressed at 5 tons for 5 minutes to form a pellet. The pellet was ground using a Waring blender. Particles were collected between a 120-mesh (125 μm) and a 400-mesh (38 μm) sieve.

### Example 6

Zinc Carbonate Particle Preparation

Particles of Zinc Carbonate hydroxide hydrate (ZnCO₃ 2Zn(OH)₂ XH₂O) (Aldrich, Milwaukee, Wis., USA) with size of 15-38 μm was prepared by sieving through 38 μm and retaining in 15 μm using 3″ stainless steel sieve.

### Example 7

Drug Loading

Particles prepared as above were added to a gel vehicle in an amount of 10-30% by weight and blended manually until the dry powder was wetted completely. Then, the milky light yellow particle/gel mixture was thoroughly blended by conventional mixing using a Caframo mechanical stirrer with an attached square-tip metal spatula. Resulting formulations are illustrated in Tables 1, 2 and 3.

TABLE 1

| | PLGA | Benzyl | Bupivacaine | |
|---|---|---|---|---|
| | RG502^{a} | Benzoate | Base | ZnCO3 |
| Formulation | (wt %) | (wt %) | (wt %) | (wt %) |
| 1 | 45 | 45 | 10 | 0 |
| 2 | 43.5 | 43.5 | 10 | 3 |

^{a}PLGA RG 502, MW = 16,000

TABLE 2

| | LMW | Benzyl | Bupivacaine | |
|---|---|---|---|---|
| | PLGA^{a} | Alcohol | HCl | ZnCO3 |
| Formulation | (wt %) | (wt %) | (wt %) | (wt %) |
| 3 | 67.5 | 22.5 | 10 | 0 |
| 4 | 65.2 | 21.8 | 10 | 3 |
| 5 | 63.0 | 21 | 10 | 6 |

^{a}Low Molecular Weight (LMW, MW = 10,000) PLGA with a carboxyl end group.

### Example 8

Co-Loading Bupivacaine Particles with Zinc Carbonate

Drug particles prepared in Example 3 were pre-mixed with Zinc Carbonate particles prepared in Example 6 with pre-determined ratios and the mixture of particles of drug and Zinc Carbonate were added to a gel vehicle in a process as described in Example 7. Resulting formulations are illustrated in Tables 1 and 2.

### Example 9

Co-Loading hGH/Zn Complex Particles with Zinc Carbonate

Particles of hGH/Zn complex prepared in Example 5 and Zinc Carbonate particles prepared in Example 6 were added to a gel vehicle separately with a pre-determined ratios and particles of hGH/Zn complex and Zinc Carbonate were mixed in a gel vehicle in a process as described in Example 7. Resulting formulations are illustrated in Table 3.

TABLE 3

| | PLGA | Benzyl | HGH/Zn | |
|---|---|---|---|---|
| | RG502^{a} | Benzoate | complex | ZnCO3 |
| Formulation | (wt %) | (wt %) | (wt %) | (wt %) |
| 6 | 45.0 | 45.0 | 10^{b} | 0 |
| 7 | 45.0 | 45.0 | 10^{c} | 0 |
| 8 | 43.5 | 43.5 | 10^{c} | 3 |

^{a}PLGA RG 502, MW = 16,000;
^{b}Particles of hGH/Zn complex was prepared without pre-compression;
^{c}Particles of hGH/Zn complex was prepared with pre-compression.

### Example 10

Bupivacaine In Vivo Studies

In vivo studies in rats (4 or 5 per group) were performed following an open protocol to determine plasma levels of bupivacaine upon systemic administration of bupivicaine via the implant systems of this invention. Depot gel bupivacaine formulations were loaded into customized 0.5 cc disposable syringes. Disposable 18 gauge needles were attached to the syringes and were heated to 37° C. using a circulator bath. Depot gel bupivacaine formulations were injected into rats and blood was drawn at specified time intervals (1 hour, 4 hours and on days 1, 2, 5, 7, 9, 14, 21 and 28) and analyzed for bupivacaine using LC/MS.

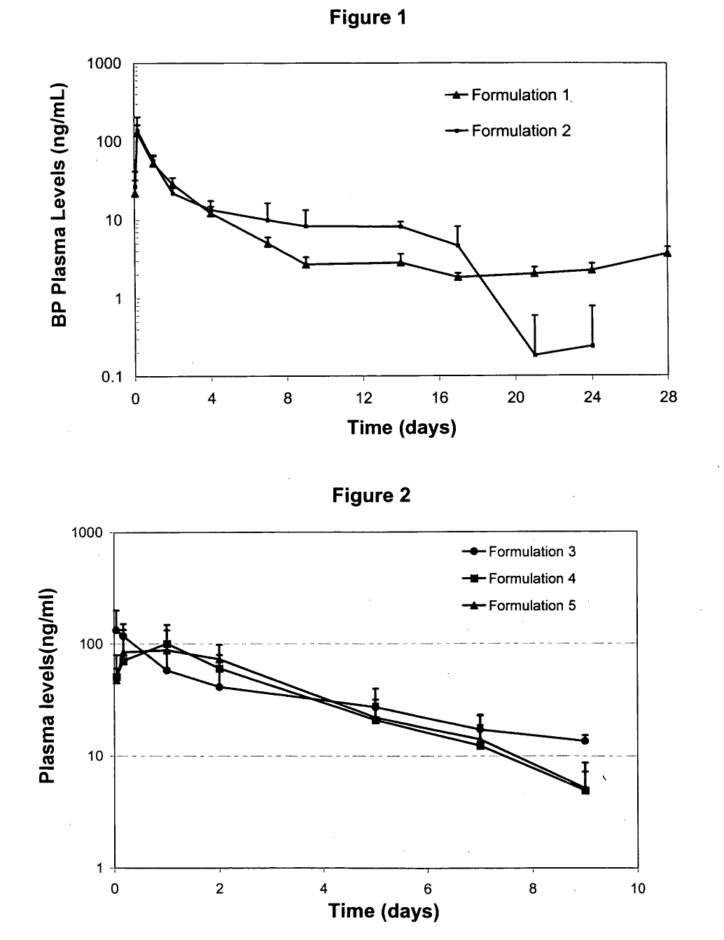

FIG. 1 illustrates the representative in vivo release profiles of bupivacaine base obtained in rats from various depot formulations for a prolonged duration system (approximately 1 month), including those of the present invention. The depot formulation without ZnCO₃ co-loaded (Formulation 1) exhibited a biphasic release profile, i.e., in the first stage (<1-2 week period), the release rate decreased with time (primarily controlled by diffusion) while in the later stage (after 1-2 weeks) the release became flat or increased over time (due to contribution of polymer degradation and diffusion). The depot formulation with ZnCO₃ co-loaded (formulation 2) did not exhibit the typical biphasic release profile, much flatter release profiles after initial burst release (as close to the one without ZnCO₃, formulation 1) and short release duration instead. This finding clearly demonstrates that the addition of ZnCO₃ into the depot formulation can alter the release rate profile from typical biphasic to near zero order release rate profiles as well as modulate the release duration.

It is surprising that the release rate shown by the depot formulation with ZnCO₃ co-loaded (formulation 2) was faster than that of the formulation without ZnCO₃ co-loaded (formulation 1). Typically, in a basic environment (pH>7.0) it is expected that bupivacaine remain in it base form and would exhibit a slow release due to its hydrophobic nature. As shown by formulation 2, however, in the presence of a weak base, e.g., ZnCO₃, (i.e., pKa>7), the release rate is faster than that without a weak base, and is similar to that exhibited by bupivacaine in a hydrophilic state.

FIG. 2 illustrates the representative in vivo release profiles of bupivacaine hydrochloride obtained in rats from various depot formulations for shorter duration system (up to 2 weeks), including those of the present invention. The depot formulation without ZnCO₃ co-loaded (Formulation 3) exhibited a release of the drug decreased over time indicating a primary diffusion controlled release profile. The depot formulation with ZnCO₃ co-loaded (formulations 4 and 5), however, exhibit reduced burst release and much flatter release profiles (near zero order) as compared to the formulation without ZnCO₃ loaded (formulation 3), indicating that the addition of ZnCO₃ into the depot formulation can also alter the release rate profile for the short duration depot.

### Example 11

hGH In Vivo Studies

In vivo studies in rats were performed following an open protocol to determine serum levels of hGH upon systemic administration of hGH via the implant systems of this invention. Depot gel hGH formulations were loaded into customized 0.5 cc disposable syringes. Disposable 18 gauge 1″ needles were attached to the syringes and were heated to 37° C. using a circulator bath. Depot gel hGH formulations were injected into immunosuppressed rats and serum samples were collected post—injection at 1 hr, 4 hr, day 1, 2, 4, 7, 10, 14; 21 and 28. All serum samples were stored at 4° C. prior to analysis. Samples were analyzed for intact hGH content using a radio immunoassay (RIA). At the end of study the rats are euthanized for gross clinical observation and the depot was retrieved for intactness observations.

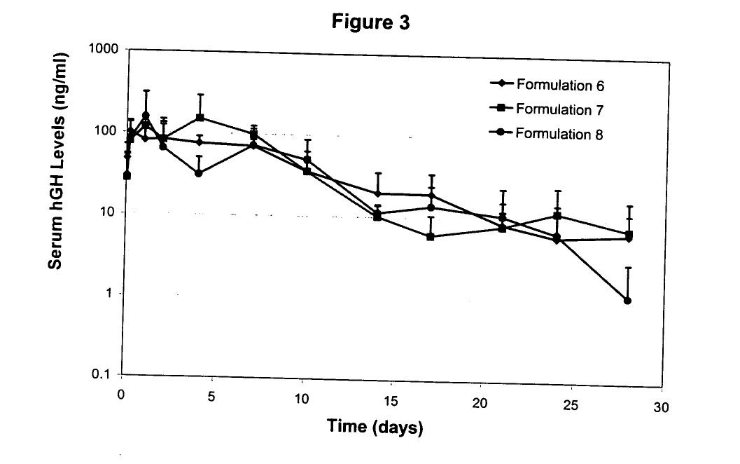

FIG. 3 illustrates representative in vivo release profiles of human growth hormone (“hGH”) obtained in rats from various depot compositions, including those of the present invention. The depot formulation with ZnCO₃ co-loaded (formulation 8) tended to have flatter release rate profile with shorter release duration as found in FIG. 1 with bupivacaine, compared with ones without ZnCO₃ co-loaded (formulations 6 and 7). This further indicates that addition of ZnCO₃ into the depot formulation as described in this invention can also alter the protein release rate profiles and modulate the release duration as well.

### Example 12

Particle Preparation of Reducing Agent

Particles of methionine, a reducing agent (Sigma, St. Louis, Mo., USA) with size of 15-38 μm are prepared by sieving through 38 μm and retaining in 15 μm using 3″ stainless steel sieve.

### Example 13

Loading of hGH and Methionine into Depot and In vivo Testing

Reducing agent, methionine, of example 12 is added to a gel vehicle in an amount of 0.1-20% by weight and is blended manually until the dry powder is wetted completely. Then, the milky light yellow particle/gel mixture is thoroughly blended by conventional mixing using a Caframo mechanical stirrer with an attached square-tip metal spatula. A therapeutic agent, such as a protein like hGH or a small molecule such as bupivacaine is loaded into the gel vehicle. The ratio of methionine to therapeutic agent is between about 0.1:99.9 to about 70:30. In vivo testing is conducted to produce release rate profiles.

### Example 14

Particle Preparation of Antioxidant

Particles of vitamin E acid succinate, an antioxidant agent, (Sigma, St. Louis, Mo., USA) with size of 15-38 μm are prepared by sieving through 38 μm and retaining in 15 μm using 3″ stainless steel sieve.

### Example 15

Drug Loading and In vivo Testing

Antioxidant, vitamin E, of example 14 is added to a gel vehicle in an amount of 0.1-20% by weight and is blended manually until the dry powder is wetted completely. Then, the milky light yellow particle/gel mixture is thoroughly blended by conventional mixing using a Caframo mechanical stirrer with an attached square-tip metal spatula. When the amount of vitamin E is low (between about 0.1 to about 5% by weight), it is dissolved in the gel vehicle. A therapeutic agent, such as a protein like hGH or a small molecule drug such as bupivacaine is loaded into the gel vehicle. The ratio of vitamin E to therapeutic agent is between about 0.1:99.9 and about 70:30. In vivo testing is conducted to produce release rate profiles

1. An injectable depot gel composition for the sustained delivery of a beneficial agent comprising:
a gel vehicle comprising a bioerodible, biocompatible polymer and a water-immiscible solvent in an amount effective to plasticize the polymer and form a gel therewith;
a beneficial agent dissolved or dispersed in the gel vehicle; and
an excipient comprising an antioxidant for modulating a release rate and stabilizing the beneficial agent;
wherein the sustained delivery occurs during a period of between about twenty-four hours to about twelve months after administration.

2. The composition of claim 1 wherein the excipient offsets the effects of peroxides or free radicals or both.

3. The composition of claim 1 further comprising a pH modifier.

4. The composition of claim 3 where the pH modifier is selected from the group consisting of: an inorganic salt, an organic salt, and combinations thereof.

5. The composition of claim 4 wherein the pH modifier is selected from the group consisting of: zinc carbonate, magnesium carbonate, calcium carbonate, magnesium hydroxide, calcium hydrogen phosphate, calcium acetate, calcium hydroxide, calcium lactate, calcium maleate, calcium oleate, calcium oxalate, calcium phosphate, magnesium acetate, magnesium hydrogen phosphate, magnesium phosphate, magnesium lactate, magnesium maleate, magnesium oleate, magnesium oxalate, zinc acetate, zinc hydrogen phosphate, zinc phosphate, zinc lactate, zinc maleate, zinc oleate, zinc oxalate, and combinations thereof.

6. The composition of claim 1 wherein the antioxidant comprises a reducing agent which comprises cysteine or methionine.

7. The composition of claim 1 wherein the antioxidant comprises a free radical scavenger.

8. The composition of claim 1 where in the antioxidant is selected from the group consisting of: d-alpha tocopherol acetate, dl-alpha tocopherol, ascorbyl palmitate, butylated hydroxyanidole, ascorbic acid, butylated hydroxyanisole, butylatedhydroxyquinone, butylhydroxyanisol, hydroxycomarin, butylated hydroxytoluene, cephalm, ethyl gallate, propyl gallate, octyl gallate, lauryl gallate, propylhydroxybenzoate, trihydroxybutylrophenone, dimethylphenol, diterlbulylphenol, vitamin E, lecithin, ethanolamine, and combinations thereof.

9. The composition of claim 1 comprising between about 0.01% and about 50% by weight of excipient.

10. The composition of claim 9 comprising between about 0.05% and about 40% by weight of excipient.

11. The composition of claim 10 comprising between about 0.1% and about 30% by weight of excipient.

12. The composition of claim 1 wherein the ratio between the excipient and the beneficial agent is between about 0.1:99.9 and about 99:1.

13. The composition of claim 12 wherein the ratio is between about 1:99 and about 60:40.

14. The composition of claim 1 wherein the solvent has a miscibility in water of less than or equal to about 7 weight % at 25° C.

15. The composition of claim 1 wherein the composition is free of solvents having a miscibility in water that is greater than 7 weight % at 25° C.

16. The composition of claim 1 wherein the solvent is selected from the group consisting of:
an aromatic alcohol, lower alkyl esters of aryl acids, lower aralkyl esters of aryl acids; aryl ketones, aralkyl ketones, lower alkyl ketones, lower alkyl esters of citric acid, and combinations thereof.

17. The composition of claim 1 wherein the solvent comprises benzyl alcohol.

18. The composition of claim 1 wherein the solvent comprises benzyl benzoate.

19. The composition of claim 1 wherein the solvent comprises ethyl benzoate.

20. The composition of claim 1 wherein the solvent comprises triacetin.

21. The composition of claim 1 wherein the solvent comprises a component solvent selected from the group consisting of: triacetin, diacetin, tributyrin, triethyl citrate, tributyl citrate, acetyl triethyl citrate, acetyl tributyl citrate, triethylglycerides, triethyl phosphate, diethyl phthalate, diethyl tartrate, mineral oil, polybutene, silicone fluid, glylcerin, ethylene glycol, polyethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, methyl acetate, ethyl acetate, methyl ethyl ketone, dimethylformamide, dimethyl sulfoxide, tetrahydrofuran, caprolactam, decylmethylsulfoxide, oleic acid, and 1-dodecylazacyclo-heptan-2-one, and combinations thereof.

22. The composition of claim 1 wherein the polymer comprises a lactic acid-based polymer.

23. The composition of claim 22 wherein the polymer comprises a copolymer of lactic acid and glycolic acid (PLGA).

24. The composition of claim 23 wherein the polymer has a weight average molecular weight of between about 3,000 to about 120,000 and the copolymer has a monomer ratio of lactic acid to glycolic acid between about 50:50 to about 100:0.

25. The composition of claim 23 wherein the polymer comprises poly(D,L-lactide-co-glycolide).

26. The composition of claim 23 wherein the polymer comprises poly(L-lactide-co-glycolide).

27. The composition of claim 1 wherein the polymer comprises a caprolactone-based polymer.

28. The composition of claim 1 wherein the polymer is selected from the group consisting of: polylactides, polyglycolides, poly(caprolactone), polyanhydrides, polyamines, polyesteramides, polyorthoesters, polydioxanones, polyacetals, polyketals, polycarbonates, polyphosphoesters, polyesters, polybutylene terephthalate, polyorthocarbonates, polyphosphazenes, succinates, poly(malic acid), poly(amino acids), polyvinylpyrrolidone, polyethylene glycol, polyhydroxycellulose, polysaccharides, chitin, chitosan, hyaluronic acid, and copolymers, terpolymers and mixtures thereof.

29. The composition of claim 1 comprising between about 5 weight % and about 90 weight % of the polymer.

30. The composition of claim 29 comprising between about 25 weight % and about 80 weight % of the polymer.

31. The composition of claim 30 comprising between about 35 weight % and about 75 weight % of the polymer.

32. The composition of claim 1 wherein the composition comprises from about 0.1% to about 50% beneficial agent by weight.

33. The composition of claim 32 wherein the composition comprises from about 0.5% to about 40% beneficial agent by weight.

34. The composition of claim 33 wherein the composition comprises from about 1% to about 30% beneficial agent by weight.

35. The composition of claim 1 wherein the ratio between the polymer and the solvent is between about 5:95 and about 90:10.

36. The composition of claim 35 wherein the ratio between the polymer and the solvent is between about 20:80 and about 80:20.

37. The composition of claim 36 wherein the ratio between the polymer and the solvent is between about 30:70 and about 75:25.

38. The composition of claim 1 further comprising at least one of the following: an emulsifying agent, a pore former, a solubility modulator for the anesthetic, and an osmotic agent.

39. The composition of claim 1 wherein the beneficial agent comprises particles having an average particle size of less than about 250 μm.

40. The composition of claim 39 wherein the average particle size is between about 5 μm and 250 μm.

41. The composition of claim 40 wherein the average particle size is between about 20 μm and about 125 μm.

42. The composition of claim 41 wherein the average particle size is between about 38 μm and about 63 μm.

43. The composition of claim 1 wherein the beneficial agent is selected from the group consisting of: a protein, a peptide, a drug, and combinations thereof.

44. The composition of claim 43 wherein the beneficial agent comprises a protein selected from the group consisting of: human growth hormone, interferon alpha-2a, interferon alpha-2b, EPO, methionine-human growth hormone, des-phenylalanine human growth hormone, consensus interferon, and combinations thereof.

45. The composition of claim 43 wherein the beneficial agent comprises a drug comprising bupivacaine or praclitaxil.

46. The composition of claim 43 wherein the beneficial agent comprises a peptide comprising leuprolide or desmopressin.

47. A method of preparing an injectable depot gel composition for sustained delivery of a beneficial agent to a subject over a duration of between about twenty-four hours to about twelve months comprising:
mixing a bioerodible, biocompatible polymer and an effective plasticizing amount of a water-immiscible solvent to form a gel vehicle;
dissolving or dispersing a beneficial agent into the gel vehicle;
mixing an excipient comprising an antioxidant for modulating a release rate into the gel vehicle; and
stabilizing the beneficial agent.

48. The method of claim 47 further comprising premixing the excipient with the beneficial agent before mixing the excipient and the beneficial agent into the gel vehicle.

49. The method of claim 48 further comprising loading the excipient and the beneficial agent separately into the gel vehicle.

50. The method of claim 47 wherein the excipient is dissolved or dispersed in the gel vehicle.

51. The method of claim 47 wherein the antioxidant offsets the effects of peroxide or free radicals or both.

52. The method of claim 47 wherein the antioxidant comprises a reducing agent which comprises cysteine or methionine.

53. The method of claim 47 wherein the antioxidant comprises a free radical scavenger.

54. The method of claim 47 where in the antioxidant is selected from the group consisting of: d-alpha tocopherol acetate, dl-alpha tocopherol, ascorbyl palmitate, butylated hydroxyanidole, ascorbic acid, butylated hydroxyanisole, butylatedhydroxyquinone, butylhydroxyanisol, hydroxycomarin, butylated hydroxytoluene, cephalm, ethyl gallate, propyl gallate, octyl gallate, lauryl gallate, propylhydroxybenzoate, trihydroxybutylrophenone, dimethylphenol, diterlbulylphenol, vitamin E, lecithin, ethanolamine, and combinations thereof.

55. The method of claim 47 further comprising loading the composition with between about 0.01% and about 50% by weight of excipient.

56. The method of claim 47 further comprising loading the excipient and the beneficial agent in a ratio of between about 0.1:99.9 and about 99:1.

57. The method of claim 56 wherein the ratio is between about 1:99 and about 60:40.

58. The method of claim 47 wherein the solvent has a miscibility in water of less than or equal to about 7 weight % at 25° C.

59. The method of claim 47 wherein the composition is free of solvents having a miscibility in water that is greater than 7 weight % at 25° C.

60. The method of claim 47 wherein the polymer comprises a lactic acid-based polymer.

61. The method of claim 60 wherein the polymer comprises a copolymer of lactic acid and glycolic acid (PLGA).

62. The method of claim 60 wherein the polymer has a weight average molecular weight of between about 3,000 to about 120,000 and the copolymer has a monomer ratio of lactic acid to glycolic acid between about 100:0 to about 15:85.

63. The method of claim 61 wherein the polymer comprises poly(D,L-lactide-co-glycolide).

64. The method of claim 61 wherein the polymer comprises poly(L-lactide-co-glycolide).

65. The method of claim 47 further comprising loading the composition with between about 5 weight % and about 90 weight % of the polymer.

66. The method of claim 47 further comprising loading the composition with between about 0.1 weight % to about 50 weight % beneficial agent.

67. A method of administering an injectable depot composition for sustained release of a beneficial agent over a duration of between about twenty-four hours to about twelve months comprising:
administering a composition comprising a gel vehicle comprising a bioerodible, biocompatible polymer and an effective plasticizing amount of a water-immiscible solvent to form a gel vehicle; a beneficial agent dissolved or dispersed in the gel vehicle; and an excipient comprising an antioxidant for modulating a release rate and stabilizing the beneficial agent.

68. The method of claim 67 further comprising administering the composition once.

69. The method of claim 67 further comprising delivering the composition locally.

70. The method of claim 67 further comprising delivering the composition systemically.

71. The method of claim 67 further comprising delivering the composition to multiple sites.

72. The method of claim 67 further comprising repeating the administration of the composition.

73. A kit for administration of a sustained delivery of a beneficial agent for a period of between about twenty-four hours to about twelve months after administration, the kit comprising:
a gel vehicle comprising a bioerodible, biocompatible polymer and a water-immiscible solvent, in an amount effective to plasticize the polymer and form a gel therewith;
a beneficial agent dissolved or dispersed in the gel vehicle;
an excipient comprising an antioxidant for modulating a release rate and for stabilizing the beneficial agent;
and optionally, one or more of the following:
a pH modifier;
an emulsifying agent;
a pore former;
a solubility modulator for the anesthetic, optionally associated with the beneficial agent; and
an osmotic agent;
wherein at the least anesthetic agent, optionally associated with the solubility modulator, is maintained separated from the solvent until the time of administration of the anesthetic agent to the subject.
